SEQUENCE LISTING (14 sequences)

SEQ ID NO: 1 - DNA, 3891 bp, Clostridium botulinum
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 3876 bp, Clostridium botulinum
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaagctccaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattttaaataatattatcttaaatttaagatataaggataataatttaatagatttatcaggatatggggcaaaggtagaggtatatgatggagtcgagcttaatgataaaaatcaatttaaattaactagttcagcaaatagtaagattagagtgactcaaaatcagaatatcatatttaatagtgtgttccttgattttagcgttagcttttggataagaatacctaaatataagaatgatggtatacaaaattatattcataatgaatatacaataattaattgtatgaaaaataattcgggctggaaaatatctattaggggtaataggataatatggactttaattgatataaatggaaaaaccaaatcggtattttttgaatataacataagagaagatatatcagagtatataaatagatggttttttgtaactattactaataatttgaataacgctaaaatttatattaatggtaagctagaatcaaatacagatattaaagatataagagaagttattgctaatggtgaaataatatttaaattagatggtgatatagatagaacacaatttatttggatgaaatatttcagtatttttaatacggaattaagtcaatcaaatattgaagaaagatataaaattcaatcatatagcgaatatttaaaagatttttggggaaatcctttaatgtacaataaagaatattatatgtttaatgcggggaataaaaattcatatattaaactaaagaaagattcacctgtaggtgaaattttaacacgtagcaaatataatcaaaattctaaatatataaattatagagatttatatattggagaaaaatttattataagaagaaagtcaaattctcaatctataaatgatgatatagttagaaaagaagattatatatatctagatttttttaatttaaatcaagagtggagagtatatacctataaatattttaagaaagaggaagaaaaattgtttttagctcctataagtgattctgatgagttttacaatactatacaaataaaagaatatgatgaacagccaacatatagttgtcagttgctttttaaaaaagatgaagaaagtactgatgagataggattgattggtattcatcgtttctacgaatctggaattgtatttgaagagtataaagattatttttgtataagtaaatggtacttaaaagaggtaaaaaggaaaccatataatttaaaattgggatgtaattggcagtttattcctaaagatgaagggtggactgaataa

SEQ ID NO: 4 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 5 - DNA, 3843 bp, Clostridium botulinum
atgccaataacaattaacaactttaattattcagatcctgttgataataaaaatattttatatttagatactcatttaaatacattagctaatgagcctgaaaaagcctttcgcattatagggaatatatgggtaatacccgatagattttcaagagattctaatccaaatttaaataaacctcctcgagttacaagccctaaaagtggttattatgatcctaattatttgagtactgattctgaaaaagatacatttttaaaagaaattataaagttatttaaaagaattaactctagagaaataggagaagaattaatatatagacttgcaacagacataccctttcctgggaataacaatactccaattaatacttttgattttgatgtagattttaacagtgttgatgttaaaactagacaaggtaacaactgggttaaaactggtagtataaatcctagtgttataataactggacctagagaaaacattatagacccagaaacttctacgtttaaattaactaacaatacttttgcggcacaagaaggatttggtgctttatcaataatttcaatatcacctagatttatgctaacatatagtaatgcaactaataatgtaggagagggtagattttctaagtctgaattttgcatggatccaatactaattttaatgcatgaacttaatcatgcaatgcataatttatatggaatagctataccaaatgatcaaagaatttcatctgtaactagtaatattttttattctcaatataaggtgaaattagagtatgcagaaatatatgcatttggaggtccaactatagaccttattcctaaaagtgcaaggaaatattttgaggaaaaggcattggattattatagatccatagctaaaagacttaatagtataactactgcaaatccttcaagctttaataaatatataggagaatataaacagaaacttattagaaagtatagattcgtagtagaatcttcaggtgaagttgcagtagatcgtaataagtttgctgagttatataaagaacttacacaaatatttacagaatttaactacgctaaaatatataatgtacaaaataggaaaatatatctttcaaatgtatatactccggttacggcaaatatattagacgataatgtttatgatatacaaaatggatttaacatacctaaaagtaatttaaatgtactatttatgggtcaaaatttatctcgaaatccagcattaagaaaagtcaatcctgaaaatatgctttatttatttacaaaattttgccataaagcaatagatggtagatcattatataataaaacattagattgtagagagcttttagttaaaaatactgacttaccctttataggtgatattagtgatatcaaaactgatatatttttaagcaaagatattaatgaagaaactgaagttatagactatccggacaatgtttcagtggatcaagttattctcagtaagaatacctcagaacatggacaactagatttattataccctattattgaaggtgagagtcaagtattaccgggagagaatcaagtcttttatgataatagaactcaaaatgttgattatttgaattcttattattacctagaatctcaaaaactaagtgataatgttgaagattttacttttacgacatcaattgaggaagctttggataatagtggaaaagtatatacttactttcctaaactagctgataaagtaaatacgggtgttcaaggtggtttatttttaatgtgggcaaatgatgtagttgaagattttactacaaatattctaagaaaagatacattagataaaatatcagatgtatcagctattattccctatataggacctgcattaaatataagtaattctgtaagaaggggaaattttactgaagcatttgcagttaccggtgtaactattttattagaagcgtttcaagaatttacaatacctgcacttggtgcatttgtgatttatagtaaggttcaagaaagaaacgagattattaaaactatagataattgtttagaacaaaggattaaaagatggaaagattcatatgaatggatgataggaacgtggttatccaggattactactcaatttaataatataagttatcaaatgtatgattctttaaattatcaggcagatgcaatcaaagataaaatagatttagaatataaaaaatactcaggaagtgataaagaaaatataaaaagtcaagttgaaaatttaaaaaatagtttagatataaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatataattaatgaatatttcaatagtattaatgattcaaaaattttgagcttacaaaacaaaaaaaatgctttagtggatacatcaggatataatgcagaagtgaggctagaaggtgatgttcaagttaatacgatatatacaaatgattttaaattaagtagttcaggagataaaattatagtaaatttaaataataatattttatatagcgctatttatgagaactctagtgttagtttttggattaagatatctaaagatttaactaattctcataatgaatatacaataattaatagtataaaacaaaattctgggtggaaattatgtattaggaatggcaatatagaatggattttacaagatattaatagaaagtataaaagtttaatttttgattatagtgaatcattaagtcatacaggatatacaaataaatggttttttgttactataactaataatataatggggtatatgaaactttatataaatggagaattaaagcagagtgaaagaattgaagatttaaatgaggttaagttagataaaaccatagtatttggaatagatgagaatatagatgagaatcagatgctttggattagagattttaatattttttctaaagaattaagcaatgaagatattaatattgtatatgagggacaaatattaagaaatgttattaaagattattggggaaatcctttgaagtttgatacagaatattatattattaatgataattatatagataggtatatagcacctaaaagtaatatacttgtacttgttcagtatccagatagatctaaattatatactggaaatcctattactattaaatcagtatctgataagaatccttatagtagaattttaaatggagataatataatgtttcatatgttatataatagtgggaaatatatgataataagagatactgatacaatatatgcaatagaaggaagagagtgttcaaaaaattgtgtatatgcattaaaattacagagtaatttaggtaattatggtataggtatatttagtataaaaaatattgtatctcaaaataaatattgtagtcaaattttctctagttttatgaaaaatacaatgcttctagcagatatatataaaccttggagattttcttttgaaaatgcatacacgccagttgcagtaactaattatgagacaaaactattatcaacttcatctttttggaaatttatttctagggatccaggatgggtagagtaa

SEQ ID NO: 6 - protein, 1280 aa, Clostridium botulinum
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYKVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLNEVKLDKTIVFGIDENIDENQMLWIRDFNIFSKELSNEDINIVYEGQILRNVIKDYWGNPLKFDTEYYIINDNYIDRYIAPKSNILVLVQYPDRSKLYTGNPITIKSVSDKNPYSRILNGDNIMFHMLYNSGKYMIIRDTDTIYAIEGRECSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 7 - DNA, 3858 bp, Clostridium botulinum
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcctagacctacttcaaagtatcaaagttattatgatcctagttatttatctactgatgagcaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattgtatggaataaatataccatctgataaaaggattcgtccacaagttagcgagggatttttttctcaagatggacccaacgtacaatttgaggaattatacacatttggaggatcagatgttgaaataatacctcaaattgaaagattacaattaagagaaaaagcattaggtcactataaagatatagcgaaaagacttaataatattaataaaactattccttctagttggagtagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaagcattatctacctgtatttgcaaatatattagatgataatatttatactataataaacggttttaatttaacaactaaaggttttaatatagaaaattcgggtcagaatatagaaaggaatcctgcactacaaaaacttagttcagaaagtgtagtagatttgtttacaaaagtatgtttaagattaacaagaaatagtagagatgattcaacatgtattcaagttaaaaataatacattaccttatgtagctgataaagatagcatttcacaagaaatatttgaaagtcaaattattacagatgagactaatgtagaaaattattcagataatttttcattagatgaatctattttagatgcaaaagtccctactaatcctgaagcagtagatccactgttacccaatgttaatatggaacctttaaatgttccaggtgaagaagaagtattttatgatgatattactaaagatgttgattatttaaactcttattattatttggaagcccaaaaattaagtaataatgttgaaaatattactcttacaacttcagttgaagaagcattaggttatagcaataagatatacacatttttacctagcttagctgaaaaagtgaataaaggtgttcaagcaggtttattcttaaattgggcgaatgaagtagttgaggattttactacaaatattatgaaaaaagatacattggataaaatatcagatgtatcagccataattccatatataggacctgccttaaatataggaaattcagcattaaggggaaactttaagcaagcatttgcaacagctggtgtagcttttttgttagaaggatttccagagtttacaatacctgcactcggtgtatttaccttttatagttctattcaagaaagagagaaaattattaaaactatagaaaattgtttagaacaaagagttaagagatggaaagattcatatcaatggatggtatcaaattggttgtcaagaattactactcgatttaatcatataagttatcaaatgtatgattctttgagttatcaggcagatgcaatcaaagctaaaatagatttagaatataaaaaatactcaggaagtgataaagaaaatataaaaagtcaagttgaaaatttaaaaaatagtttagatgtaaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatatgattaatgaatatttcaatagtattaatgattcaaaaattttgagcttacaaaataaaaaaaatactttgatggatacatcaggatataacgcagaagtgagagtagaaggcaatgttcagcttaatccaatatttccatttgactttaaattaggtagttcaggggatgatagaggtaaagttatagtaacccagaatgaaaatattgtatataatgctatgtatgaaagttttagtattagtttttggattaggataaataaatgggtaagtaatttacctggatatactataattgatagtgttaaaaataactcaggttggagtataggtattattagtaattttttagtgtttactttaaaacaaaatgaaaatagtgaacaagatataaactttagttatgatatatcaaagaatgctgcgggatataataaatggttttttgtaactattactaccaatatgatgggaaatatgatgatttatataaatggaaaattaatagatactataaaagttaaagagttaactggaattaattttagcaaaactataacatttcaaatgaataaaattccaaatactggcttaattacctcagattctgataacatcaatatgtggataagggatttttatatctttgctaaagaattagatgataaagatattaatatattatttaatagcttgcaatatactaatgttgtaaaagattattggggaaatgatttaagatatgataaagaatattacatgattaacgtaaattatatgaatagatatatgtctaaaaaaggcaatggaattgtttttaatacacgtaaaaataataatgacttcaatgaaggatataaaattataataaaaagaattagaggaaatacaaatgatactagagtacgaggagaaaatgtattatattttaatactacaattgataacaaacaatatagtttaggtatgtataaaccttctagaaatctagggactgatttagttccactaggtgcattggatcaaccaatggatgagatacgtaaatatggttcgtttataatacaaccatgcaatacttttgattactatgcatcacaattatttttgtcaagtaatgcaacaacaaataggcttggaatactatcaattggtagttatagtttcaaacttggagatgactattggtttaatcacgaatatttaattcctgttataaaaatagagcattatgcttcattattagaatcaacatcaactcattgggtttttgtacctgcaagtgaataa

SEQ ID NO: 8 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTRFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIRGNTNDTRVRGENVLYFNTTIDNKQYSLGMYKPSRNLGTDLVPLGALDQPMDEIRKYGSFIIQPCNTFDYYASQLFLSSNATTNRLGILSIGSYSFKLGDDYWFNHEYLIPVIKIEHYASLLESTSTHWVFVPASE

SEQ ID NO: 9 - DNA, 3756 bp, Clostridium botulinum
atgccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcaccgttttggatcaatagctatagtaacattctcacctgaatattcttttagatttaatgataattgtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttacgaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggaaatcaatatgtatcgaaataaataatggtgagttattttttgtggcttccgagaatagttataatgatgataatataaatactcctaaagaaattgacgatacagtaacttcaaataataattatgaaaatgatttagatcaggttattttaaattttaatagtgaatcagcacctggactttcagatgaaaaattaaatttaactatccaaaatgatgcttatataccaaaatatgattctaatggaacaagtgatatagaacaacatgatgttaatgaacttaatgtatttttctatttagatgcacagaaagtgcccgaaggtgaaaataatgtcaatctcacctcttcaattgatacagcattattagaacaacctaaaatatatacatttttttcatcagaatttattaataatgtcaataaacctgtgcaagcagcattatttgtaagctggatacaacaagtgttagtagattttactactgaagctaaccaaaaaagtactgttgataaaattgcagatatttctatagttgttccatatataggtcttgctttaaatataggaaatgaagcacaaaaaggaaattttaaagatgcacttgaattattaggagcaggtattttattagaatttgaacccgagcttttaattcctacaattttagtattcacgataaaatcttttttaggttcatctgataataaaaataaagttattaaagcaataaataatgcattgaaagaaagagatgaaaaatggaaagaagtatatagttttatagtatcgaattggatgactaaaattaatacacaatttaataaaagaaaagaacaaatgtatcaagctttacaaaatcaagtaaatgcaattaaaacaataatagaatctaagtataatagttatactttagaggaaaaaaatgagcttacaaataaatatgatattaagcaaatagaaaatgaacttaatcaaaaggtttctatagcaatgaataatatagacaggttcttaactgaaagttctatatcctatttaatgaaaataataaatgaagtaaaaattaataaattaagagaatatgatgagaatgtcaaaacgtatttattgaattatattatacaacatggatcaatcttgggagagagtcagcaagaactaaattctatggtaactgataccctaaataatagtattccttttaagctttcttcttatacagatgataaaattttaatttcatattttaataaattctttaagagaattaaaagtagttcagttttaaatatgagatataaaaatgataaatacgtagatacttcaggatatgattcaaatataaatattaatggagatgtatataaatatccaactaataaaaatcaatttggaatatataatgataaacttagtgaagttaatatatctcaaaatgattacattatatatgataataaatataaaaattttagtattagtttttgggtaagaattcctaactatgataataagatagtaaatgttaataatgaatacactataataaattgtatgagagataataattcaggatggaaagtatctcttaatcataatgaaataatttggacattcgaagataatcgaggaattaatcaaaaattagcatttaactatggtaacgcaaatggtatttctgattatataaataagtggatttttgtaactataactaatgatagattaggagattctaaactttatattaatggaaatttaatagatcaaaaatcaattttaaatttaggtaatattcatgttagtgacaatatattatttaaaatagttaattgtagttatacaagatatattggtattagatattttaatatttttgataaagaattagatgaaacagaaattcaaactttatatagcaatgaacctaatacaaatattttgaaggatttttggggaaattatttgctttatgacaaagaatactatttattaaatgtgttaaaaccaaataactttattgataggagaaaagattctactttaagcattaataatataagaagcactattcttttagctaatagattatatagtggaataaaagttaaaatacaaagagttaataatagtagtactaacgataatcttgttagaaagaatgatcaggtatatattaattttgtagccagcaaaactcacttatttccattatatgctgatacagctaccacaaataaagagaaaacaataaaaatatcatcatctggcaatagatttaatcaagtagtagttatgaattcagtaggaaattgtacaatgaattttaaaaataataatggaaataatattgggttgttaggtttcaaggcagatactgtcgttgctagtacttggtattatacacatatgagagatcatacaaacagcaatggatgtttttggaactttatttctgaagaacatggatggcaagaaaaataa

SEQ ID NO: 10 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 11 - DNA, 3843 bp, Clostridium botulinum
atgccagttgtaataaatagttttaattataatgaccctgttaatgatgagacaattttatacatgcagaaaccatatgaagaaagaagtagaaaatattataaagcttttgagattatgcctaatgtttggataatgcctgagagagatacaataggaactaagcctgatgagtttcaggtgccggattcattaaagaacggaagtagtgcttattatgatcctaattatttaaccactgatgctgaaaaagatagatatttaaaaacaatgataaaattatttaatagaattaatagtaatcctacagggaaagttttgttagaagaagtatcaaatgctagaccatatttaggagatgatgacacgctaattaatgaattccttccagttaatgtaactacaagtgttaatataaaattttcaactgatgttgaaagttcaataatatcgaatcttcttgtattgggagcaggacctgatatatttaaagcttactgtaccccccttgtaaggtttaataagtcagataaattaattgaaccaagtaatcatggttttggatcaattaatatcttgacattttcacctgagtatgaacatatttttaatgatattagtggagggaatcataatagtacagaatcatttattgcagatcctgcaatttcactagctcatgaattgatacatgcactacatggattatacggggctaaggcagttactcataaagagtctctagtagcagagcgaggacctcttatgatagccgaaaagcccataaggctagaagaatttttaacttttggaggtgaggatttaaatatcattcctagtgctatgaaggaaaaaatatataacgatcttttagctaactatgaaaaaatagctactagacttagagaagttaatacggctcctcctggatatgatattaatgaatataaagattattttcaatggaagtatggactagatagaaatgcagatggaagttatactgtgaatagaaataaatttaatgaaatttataaaaaattatatagctttacagagattgacttagcaaataaatttaaagtaaaatgtagaaatacttattttattaaatatggatttgtaaaagttccaaatttgttagatgatgatatttatactgtatcagaggggtttaatataggtaatttagcagtaaacaatcgcggacaaaatataaatttaaatcctaaaattattgattccattccagataaaggtttagtggaaaagattattaaattttgtaagagcattattcctagaaaaggtacgaagcagtcaccgtcactatgcattagagtaaataatagggagttattttttgtagcttcagaaagtagctataatgaaagtgatattaatacacctaaagaaattgacgatacaacaaatctaaataataattatagaaataatttagatgaagttattttagattataatagtgagacaatacctcaaatatcaaatcgaacattaaatacacttgtacaagacaatagttatgtgccaagatatgattctaatggaacaagtgaaatagaggaatatgatgttgttgactttaatgtatttttctatttacatgcacaaaaagtaccagaaggtgaaaccaatataagtttaacttcttcaattgatacagcattattagaagaatccaaagtatatacatttttttcttcagagtttatcgatactatcaataaacctgtaaatgcagcactatttatagattggataagcaaagtaataagagattttaccactgaagctacacaaaaaagtactgttgataagattgcagacatatctttaattgtaccctatgtaggtcttgctttgaatatagttattgaggcagaaaaaggaaattttgaggaggcatttgaattattaggagcgggtattttattagaatttgtgccagagcttacaattcctgtaattttagtgtttacgataaaatcctatatagattcatatgagaataaaaataaagcaattaaagcaataaataattcattaatcgaaagagaagcaaagtggaaagaaatatatagttggatagtatcaaattggcttactagaattaatacgcaatttaataaaagaaaagagcaaatgtatcaggctttacaaaatcaagtagatgcaataaaaacagcaatagaatataaatataataattatacttcagatgagaaaaatagacttgaatctaaatataatatcaataatatagaagaagaattgaataaaaaagtttctttagcaatgaaaaatatagaaagatttatgacagaaagttctatatcttatttaatgaaattaataaatgaagccgaagttggtaaattaaaagaatatgataaacatgttaagagcgatttattagactatattctctaccataaattaatcttaggagagcagacaaaggaattaattgatttggtgactagtactttgaatagtagtattccatttgaactttcttcatatactaatgataaaattctaattatatattttaatagattatataaaaaaattaaagatagttctattttagatatgcgatatgaaaataataaatttatagatatctctggatatggttcaaatataagcattaatggaaacgtatatatttattcaacaaatagaaatcaatttggaatatatagtggtaggcttagtgaagttaatatagctcaaaataatgatattatatacaatagtagatatcaaaattttagtattagtttctgggtaaccattcctaaacactacagacctatgaatcgtaatcgggaatacactataataaattgtatggggaataataattcgggatggaaaatatcacttagaactattagagattgtgaaataatttggactttacaagatacttccggaaataaggaaaaattaatttttaggtatgaagaacttgctagtatatctgattatataaataaatggatttttgtaactattactaataatagattaggcaattctagaatttacatcaatggaaatttaatagttgaaaaatcaatttcgaatttaggtgatattcatgttagtgataatatattatttaaaattgttggttgtgatgatgaaacgtatgttggtataagatattttaaagtttttaatacggaattagataaaacagaaattgagactttatatagtaatgagccagatccaagtatcttaaaagactattggggaaattatttgctatataataaaaaatattatttattcaatttactaagaaaagataagtatattactcggaattcaggcattttaaatattaatcaacaaagaggtgttactggaggcatatctgtttttttgaactataaattatatgaaggagtagaagttattataagaaaaaatgctcctatagatatatctaatacagataattttgttagaaaaaacgatctagcatacattaatgtagtagatcatggtgtagaatatcggttatatgctgatatatcaattacaaaatcagagaaaataataaaattaataagaacatctaatccaaacgatagcttaggtcaaattatagttatggattcaataggaaataattgcacaatgaattttcaaaacaatgatgggagcaatataggattactaggttttcattcagatgatttggttgctagtagttggtattataaccatatacgaagaaacactagcagtaatggatgcttttggagttttatttctaaagagcatggttggaaagaataa

SEQ ID NO: 12 - protein, 1280 aa, Clostridium botulinum
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFLPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPNLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVTIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 13 - DNA, 3894 bp, Clostridium botulinum
  misc_feature (20)..(20): n is a, c, g, or t
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaaaataccggtaaatctgaacagtgtattattgttaataatgaggatttatttttcatagctaataaagatagtttttcaaaagatttagctaaagcagaaactatagcatataatacacaaaataatactatagaaaataatttttctatagatcagttgattttagataatgatttaagcagtggcatagacttaccaaatgaaaacacagaaccatttacaaattttgacgacatagatatccctgtgtatattaaacaatctgctttaaaaaaaatttttgtggatggagatagcctttttgaatatttacatgctcaaacatttccttctaatatagaaaatctacaactaacgaattcattaaatgatgctttaagaaataataataaagtctatacttttttttctacaaaccttgttgaaaaagctaatacagttgtaggtgcttcactttttgtaaactgggtaaaaggagtaatagatgattttacatctgaatccacacaaaaaagtactatagataaagtttcagatgtatccataattattccctatataggacctgctttgaatgtaggaaatgaaacagctaaagaaaattttaaaaatgcttttgaaataggtggagccgctatcttaatggagtttattccagaacttattgtacctatagttggattttttacattagaatcatatgtaggaaataaagggcatattattatgacgatatccaatgctttaaagaaaagggatcaaaaatggacagatatgtatggtttgatagtatcgcagtggctctcaacggttaatactcaattttatacaataaaagaaagaatgtacaatgctttaaataatcaatcacaagcaatagaaaaaataatagaagatcaatataatagatatagtgaagaagataaaatgaatattaacattgattttaatgatatagattttaaacttaatcaaagtataaatttagcaataaacaatatagatgattttataaaccaatgttctatatcatatctaatgaatagaatgattccattagctgtaaaaaagttaaaagactttgatgataatcttaagagagatttattggagtatatagatacaaatgaactatatttacttgatgaagtaaatattctaaaatcaaaagtaaatagacacctaaaagacagtataccatttgatctttcactatataccaaggacacaattttaatacaagtttttaataattatattagtaatattagtagtaatgctattttaagtttaagttatagaggtgggcgtttaatagattcatctggatatggtgcaactatgaatgtaggttcagatgttatctttaatgatataggaaatggtcaatttaaattaaataattctgaaaatagtaatattacggcacatcaaagtaaattcgttgtatatgatagtatgtttgataattttagcattaacttttgggtaaggactcctaaatataataataatgatatacaaacttatcttcaaaatgagtatacaataattagttgtataaaaaatgactcaggatggaaagtatctattaagggaaatagaataatatggacattaatagatgttaatgcaaaatctaaatcaatatttttcgaatatagtataaaagataatatatcagattatataaataaatggttttccataactattactaatgatagattaggtaacgcaaatatttatataaatggaagtttgaaaaaaagtgaaaaaattttaaacttagatagaattaattctagtaatgatatagacttcaaattaattaattgtacagatactactaaatttgtttggattaaggattttaatatttttggtagagaattaaatgctacagaagtatcttcactatattggattcaatcatctacaaatactttaaaagatttttgggggaatcctttaagatacgatacacaatactatctgtttaatcaaggtatgcaaaatatctatataaagtattttagtaaagcttctatgggggaaactgcaccacgtacaaactttaataatgcagcaataaattatcaaaatttatatcttggtttacgatttattataaaaaaagcatcaaattctcggaatataaataatgataatatagtcagagaaggagattatatatatcttaatattgataatatttctgatgaatcttacagagtatatgttttggtgaattctaaagaaattcaaactcaattatttttagcacccataaatgatgatcctacgttctatgatgtactacaaataaaaaaatattatgaaaaaacaacatataattgtcagatactttgcgaaaaagatactaaaacatttgggctgtttggaattggtaaatttgttaaagattatggatatgtttgggatacctatgataattatttttgcataagtcagtggtatctcagaagaatatctgaaaatataaataaattaaggttgggatgtaattggcaattcattcccgtggatgaaggatggacagaataa

SEQ ID NO: 14 - protein, 1297 aa, Clostridium botulinum
  MISC_FEATURE (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

(19) United States
(12) Patent Application Publication
(10) Pub. No.: US 2021/0393748 A1
(43) Pub. Date: Dec. 23, 2021

(54) NOVEL USES OF BOTULINUM NEUROTOXIN FOR TREATING LIPOEDEMA

(71) Applicant: Merz Pharma GmbH & Co. KGaA, Frankfurt am Main (DE)

(72) Inventor: Astrid SCHESCHONKA, Frankfurt am Main (DE)

(21) Appl. No.: 17/281,849

(22) PCT Filed: Sep. 27, 2019
(86) PCT No.: PCT/EP2019/076259

§ 371 Date: Mar. 31, 2021

(30) Foreign Application Priority Data
Oct. 2, 2018 (EP) 18198238.0

(51) Int. Cl. A61K 38/48; A61P 29/00; A61K 9/00

(52) U.S. Cl. CPC A61K 38/4893; C12Y 304/24069; A61K 9/0019; A61P 29/00

(57) ABSTRACT

This invention relates to novel uses of botulinum neurotoxin in treating a disease or condition associated with lipoedema, in particular uses of botulinum neurotoxin in treating pain associated with lipoedema.

### FIELD OF THE INVENTION

This invention relates to novel uses of botulinum neurotoxins for the treatment of a disease or condition associated with lipoedema, in particular uses of botulinum neurotoxin in treating pain associated with lipoedema.

### BACKGROUND OF THE INVENTION

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TxNT) function via a similar initial physiological mechanism of action, inhibiting neurotransmitter release from the axon of the affected neuron into the synapse, they differ in their clinical response. While the botulinum neurotoxin acts at the neuromuscular junction and other cholinergic synapses in the peripheral nervous system, inhibiting the release of the neurotransmitter acetylcholine and thereby causing flaccid paralysis, the tetanus toxin, which is transcytosed into central neurons, acts mainly in the central nervous system, preventing the release of the inhibitory neurotransmitters GABA (gamma-aminobutyric acid) and glycine by degrading the protein synaptobrevin. The consequent overactivity of spinal cord motor neurons causes generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic serotypes, termed BoNT/A through BoNT/G with further subtypes. Most Clostridium botulinum strains produce one type of neurotoxin, but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial endoprotease to obtain the biologically active toxin.

The molecular mechanism of intoxication by TxNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TxNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

In Clostridium botulinum, the botulinum neurotoxin is formed as a protein complex comprising the neurotoxic component and non-toxic proteins. The accessory proteins embed the neurotoxic component thereby protecting it from degradation by digestive enzymes in the gastrointestinal tract. Thus, botulinum neurotoxins of most serotypes are orally toxic. Complexes with, for example, 450 kDa or with 900 kDa are obtainable from cultures of Clostridium botulinum.

In recent years, botulinum neurotoxins have been used as therapeutic agents, for example in the treatment of dystonias and spasms, and have additionally been used in cosmetic applications, such as for example the treatment of wrinkles like e.g. glabellar frown lines, horizontal forehead lines and lateral canthal lines. Preparations comprising botulinum toxin type A with complexing proteins are commercially available, e.g. from Ipsen Ltd (DYSPORT®/AZZALURE®) or Allergan Inc. (Botox®/VISTABEL®). In contrast to these, a highly purified neurotoxin, free from any complexing proteins, is available from Merz Pharmaceuticals GmbH, Frankfurt (Xeomin®). Further, botulinum toxin type B is marketed under the brand name MYOBLOC® (RimabotulinumtoxinB) from Solstice Neurosciences, LLC.

Clostridial neurotoxins are usually injected into the affected muscle tissue, bringing the agent close to the neuromuscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity. Immunologic tolerance to botulinum toxin has been shown to correlate with cumulative doses.

Clostridial neurotoxins display variable durations of action that are serotype specific. The clinical therapeutic effect of BoNT/A lasts approximately 3 to 4 months for neuromuscular disorders and up to 12 months for hyperhidrosis. The effects of BoNT/E, on the other hand, last about 4 weeks. One possible explanation for the divergent durations of action might be the distinct subcellular localizations of BoNT serotypes. The protease domain of BoNT/A light chain localizes in a punctate manner to the plasma membrane of neuronal cells, co-localizing with its substrate SNAP-25. In contrast, the short-duration BoNT/E serotype is cytoplasmic. Membrane association might protect BoNT/A from cytosolic degradation mechanisms allowing for prolonged persistence of BoNT/A in the neuronal cell.

The longer lasting therapeutic effect of BoNT/A makes it preferable for certain clinical uses and in particular for certain cosmetic uses compared to the other serotypes, for example serotypes B, C₁, D, E, F, G.

Lipoedema is a chronic disorder of subcutaneous tissue of unknown etiology mostly affecting post-puberty women. Males are also affected in some cases, but to a lesser extent. The disease has a negative impact on self-esteem, mobility, and quality of life. Lipoedema is typically characterized by symmetrical, disfiguring hyperplastic adipose tissue combined with bruising and pain. Untreated lipoedema fosters osteoarthritis, secondary lymphedema, limited mobility, increased risk for skin infections and psychosocial stigmatization. The adiposity is typically unresponsive to weight loss. In addition to the aesthetic deformity, patients also describe pain in the extremities, often spontaneous but also particularly upon pressure, as well as easy bruising. A differentiation of lipoedema from other conditions causing fatty excess in the lower extremities can be difficult. Diagnosing lipoedema relies on some of the characteristics of lipoedema, such as the easy bruising and the pain caused by soft tissue pressure, as well as the step-off at the ankles. Due to the incomplete understanding of the pathophysiology of lipoedema and the many unanswered questions regarding optimal therapeutic management, treatment options still remain limited. The current goals of lipoedema treatment include reducing related extremity symptoms and functional limitations and preventing progression of the disease. An etiology-directed treatment for lipoedema is currently unavailable. The standard conservative therapy for lipoedema includes, for example compression therapy, developing an active lifestyle and weight-loss programs. Compression therapy and therapeutic compression garments do not result in a decrease of fat deposition but can help to prevent further edema formation and stimulate the arterial, venous, and lymphatic flow, thereby reducing coinciding issues. Pain-relieving interventions could be useful in particular in the initial treatment phase as pain relief may increase the patients' capability to initiate a healthier and more active lifestyle. For patients with minimal or no improvement with conservative treatment, surgical options may be evaluated. Lipectomy and liposuction are used to treat lipoedema (A. Peled et al. International Journal of Women's Health 2016:8 389-395). US2009/232851, furthermore, discloses treating Adiposis dolorosa (Dercum's disease), a disease with symptoms in part similar to lipoedema, by using a combination of botulinum toxin and an opiate.

There is a strong demand to further improve the therapeutic options available for treating lipoedema. In particular it is desirable to effectively treat the pain which is associated with lipoedema. To date, such aspects have not been addressed satisfactorily.

### OBJECTS OF THE INVENTION

It was an object of the present invention to improve the treatment of lipoedema, in particular to provide a treatment for the pain which is associated with lipoedema. It was furthermore an object of the present invention to provide a treatment for pain associated with lipoedema and to avoid concomitant treatment with systemic pain treatment, like e.g. opiates.

### SUMMARY OF THE INVENTION

Surprisingly, it has been identified that botulinum neurotoxin may be used advantageously to provide a treatment of lipoedema, in particular to provide a treatment of pain which is associated with lipoedema, without the need to use an opiate or an opiate derivate. In comparison to an invasive or surgical treatment of lipoedema, the administration of botulinum neurotoxin according to the present invention enables a minimally-invasive treatment of lipoedema. Advantageously, the usage of a botulinum neurotoxin according to the invention may provide a pain alleviating effect of about 3-4 months and is therefore less burdensome for the patient in comparison to a potential systemic, e.g. oral pain treatment which requires an administration of drugs on a regular and in particular more frequent base. In addition, local pain treatment with a botulinum neurotoxin according to the invention may help the patient tolerate compression therapy better and increase the patients' capability to initiate a more active lifestyle, e.g. to do sport etc.

Thus, in one aspect, the present invention relates to a botulinum neurotoxin for use in treating a disease or condition associated with lipoedema, wherein the botulinum neurotoxin is not administered in combination with an opiate or opiate derivative.

In another aspect, the present invention relates to a pharmaceutical composition comprising a botulinum neurotoxin for use in treating a disease or condition associated with lipoedema, wherein the botulinum neurotoxin is not administered in combination with an opiate or opiate derivative.

In yet another aspect, the present invention relates to a method of treating a disease or condition associated with lipoedema, wherein the botulinum neurotoxin is not administered in combination with an opiate or opiate derivative.

In a further aspect, the present invention relates to a botulinum neurotoxin for use in treating a disease or condition associated with lipoedema, wherein the botulinum neurotoxin is administered in combination with an opiate or opiate derivative.

In another aspect, the present invention relates to a pharmaceutical composition comprising a botulinum neurotoxin for use in treating a disease or condition associated with lipoedema, wherein the botulinum neurotoxin is administered in combination with an opiate or opiate derivative.

In yet another aspect, the present invention relates to a method of treating a disease or condition associated with lipoedema, wherein the botulinum neurotoxin is administered in combination with an opiate or opiate derivative.

### DETAILED DESCRIPTION OF THE INVENTION

The present invention may be understood more readily by reference to the following detailed description of the invention and the examples included therein.

In one aspect the invention relates to a botulinum neurotoxin for use in treating a disease or condition associated with lipoedema, wherein the botulinum neurotoxin is not administered in combination with an opiate or opiate derivative.

In another aspect, the present invention relates to a botulinum neurotoxin for use in treating a disease or condition associated with lipoedema, wherein the botulinum neurotoxin is administered in combination with an opiate or opiate derivative.

In the context of the present invention, the term “botulinum neurotoxin” refers to a natural neurotoxin obtainable from bacteria Clostridium botulinum or to a neurotoxin obtainable from alternative sources, including from recombinant technologies or from genetic or chemical modification. Particularly, the botulinum neurotoxins have endopeptidase activity.

In particular embodiments, the botulinum neurotoxin is for use in treating pain associated with lipoedema. The occurrence of pain in lipoedema is not yet completely understood. Without wishing to be bound by theory, it could be that pain in lipoedema is caused by i) compression of neural structures due to lipoedema, ii) disturbance of neural function through toxic substances that are accumulated due to disease or iii) a long term disturbance of neural function (neuropathy) due to both i) and ii). However, additional causes cannot yet be excluded. Generally, pain is experienced when the free nerve endings which constitute the pain receptors in the skin as well as in certain internal tissues are subjected to mechanical or other noxious stimuli. The pain receptors can transmit signals along afferent neurons into the central nervous system and then to the brain. Ineffectively treated pain can be devastating to the person experiencing it by interfering with the quality of life. The typical oral, parenteral or topical administration of an analgesic drug to treat the symptoms of pain can result in widespread systemic distribution of the drug and undesirable side effects. There is no existing method for adequately and specifically treating established pain associated with lipoedema. Significantly, the pain alleviating effect of the present invention can persist for about 3-4 months and longer in some circumstances. In a preferred embodiment of the present invention, the botulinum neurotoxin for use in treating a disease or condition associated with lipoedema will be administered to a patient with lipoedema having intact skin and limited extent of secondary skin changes according to physician's judgement.

The term “disease or condition associated with lipoedema” refers in the context of the present invention to a disease or condition which is caused by lipoedema. In particular, the pain associated with lipoedema is predominantly the result of the lipoedema.

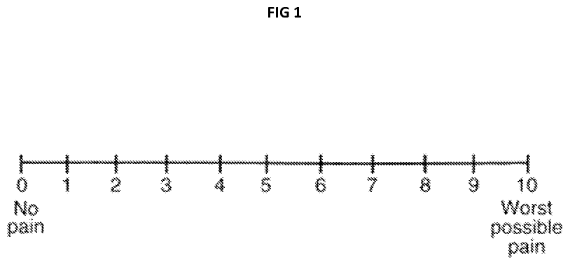

According to S. Rapprich et al. (“Treatment of lipoedema using liposuction”; Phlebologie 2015; 44: 121-132) the intensity of pain associated with lipoedema can be generally reported on a Visual Analogue Scale (VAS) from 0 (absent) to 10 (very severely pronounced). Spontaneous pain can be considered as a useful parameter which can be measured using VAS. Prior to treatment, the patients rate their pain with a score of points (0-10). About 2-4 weeks after the treatment using a botulinum neurotoxin, the pain score is again determined using VAS. As an alternative method, scales of pain can be determined as described in the publication by Schmeller et al. (Schmerzen beim Lipodem; LymphForsch 12 (1) 2008). According to that method, qualitative as well as semi-quantitative information are used to determine the degree of pain. Rapprich et al. (Journal der Deutschen Dermatologischen Gesellschaft 9(1):33-40, 2011) used a VAS to assess therapeutic effects of liposuction on lipoedema symptoms. Their assessment included questions like: “Do you have pain in the affected areas?”, “Do you have pain when experiencing pressure?”, “Do you have sensation of tension in the affected areas?”, “Do your legs feel heavy?”, “Do you observe to be prone for bruising?”, “Do you have difficulties when walking?” or “Is your quality of life impaired”. An example of VAS for pain is presented in FIG. 1.

FIG. 1: Reporting the intensity of pain on a Visual Analogue Scale (VAS)

In one embodiment of the present invention the botulinum neurotoxin for use in treating pain associated with lipoedema is administered to a patient having an intensity of pain on the visual analogue score of more than 3. In a further embodiment of the present invention the botulinum neurotoxin for use in treating pain associated with lipoedema is administered to a patient having an intensity of pain on the visual analogue score of more than 5. In a further preferred embodiment of the present invention the botulinum neurotoxin for use in treating pain associated with lipoedema is administered to a patient having an intensity of pain on the visual analogue score of more than 7.

According to a further aspect of the present invention, the VAS can also be used to assess the reduction of the quality of life of a patient having lipoedema. Using questions like “how do you score the reduction of your quality of life” (Rapprich et al. (Journal der Deutschen Dermatologischen Gesellschaft 9(1):33-40, 2011) or “how severely does the pain due to lipoedema affect your quality of life” or “how do you score the reduction of your quality of life due to the pain arising from lipoedema”. Quality of life could also be rated on a VAS by asking “how much does the pain due to lipoedema prevent you from conducting your usual daily activities” (impairment due to pain), “how severely do you worry not to be able to return to a normal life due your pain from lipoedema” (anxiety, depression), “how much does your pain due to lipoedema keep you from participating in activities with others” (isolation), “how severely does your pain due to lipoedema prevent you to have the career you are intending to have” (participation, impairment to meet challenges in worklife), “how much does the long-standing issue of pain due to lipoedema affect your personality” (changes of personality due to long-standing pain).

According to a further aspect of the present invention, pain associated with lipoedema can also be measured by using other generic scales like the EQ5D5L (five dimensions, five levels). In the description part, health status is measured in terms of five dimensions (5D); mobility, self-care, usual activities, pain/discomfort, and anxiety/depression. “Mobility dimension” asks about the person's walking ability. “Self-care dimension” asks about the ability to wash or dress by oneself, and usual activities dimension measures performance in “work, study, housework, family or leisure activities”. “Pain/discomfort dimension” asks how much pain or discomfort they have, and “anxiety/depression dimension” asks how anxious or depressed they are. The respondents self-rate their level of severity for each dimension using a five-level (EQ-5D-5L) scale: having no problems (1), having slight problems (2), having moderate problems (3), having severe problems (4) and being unable to do/having extreme problems (5). As a result, a person's health status can be defined by a 5-digit number, ranging from 11111 (having no problems in all dimensions) to 55555 (having extreme problems in all dimensions). A health status of “12521” indicates having no problems in mobility and anxiety/depression, having slight problems in self-care and pain/discomfort, and having extreme problems in usual activities. This new version can define 3,125 (=5⁵) different health states. In the evaluation part of the EQ-5D-5L, the respondents evaluate their overall health status using the visual analogue scale (EQ-VAS) (see description above). Thus, the evaluation resembles to that of the VAS but gives a larger context of quality of life. The following table depicts potential patient cases that would be selected for pain treatment with a botulinum neurotoxin according to the present invention (EQ-5D-5L, level assessment).

TABLE 1

| Selection of | | | | | |
|---|---|---|---|---|---|
| Potential | | Self- | Usual | Pain/ | Anxiety/ |
| Patient types | Mobility | care | activites | discomfort | depression |
| slight pain | 2 | 4 | 4 | 2 | 2 |
| impairing self- | | | | | |
| care and usual | | | | | |
| activities | | | | | |
| Severe pain | 5 | 5 | 5 | 5 | 5 |
| imparing all | | | | | |
| other dimensions | | | | | |
| Moderate pain | 1 | 1 | 4 | 3 | 1 |
| only affecting | | | | | |
| one dimension | | | | | |

According to a further preferred embodiment of the present invention the botulinum neurotoxin for use in treating pain associated with lipoedema is administered to a patient having an EQ-5D-5L level assessment of 24422, 55555 or 11431.

In a preferred embodiment of the present invention the disease or condition associated with lipoedema is pain. Pain associated with lipoedema differs from neuropathic pain in certain aspects. For example, neuropathic pain is characterized by the fact that a nerve is damaged, whereas the pain associated with lipoedema is not necessarily caused by a damaged nerve, especially at the beginning of the disease/condition. A further differentiating characteristic of pain associated with lipoedema is the fact that it is described as an unbearable feeling of tension which is atypical for neuropathic pain.

In particular embodiments, the botulinum neurotoxin is administered by intradermal or subcutaneous injection. In the context of the present invention, an intradermal administration refers to an injection of the botulinum neurotoxin into the dermis of the patient, without affecting the subcutaneous area. Generally, an intradermal administration injects the botulinum neurotoxin in a depth of 0.005 to 2 mm below the skin surface. In a preferred embodiment, an intradermal administration injects the botulinum neurotoxin in a depth of 0.005 to 1 mm below the skin surface. In a particular preferred embodiment, an intradermal administration injects the botulinum neurotoxin in a depth of 0.005 to 0.1 mm below the skin surface. In another particular embodiment, an intradermal administration injects the botulinum neurotoxin in a depth of 0.05 to 1 mm below the skin surface. As the dermis can have a thickness of up to 5 mm (e.g. soles of the feet) the maximum depth of intradermal injection of the neurotoxin might be even deeper. In particular embodiments the botulinum neurotoxin is administered in a depth of up to 5 mm below the skin surface. A subcutaneous injection refers in the context of the present invention to an injection of the botulinum neurotoxin through the dermis and into the subcutaneous area in the treatment area of the patient. The subcutaneous area is typically located between dermis and the next tissue layer. In most cases, the subcutaneous area is the area between dermis and fascia. In the context of the present invention, a subcutaneous administration injects the botulinum neurotoxin in a depth of 0.5 mm to several cm below the skin surface, depending on the fat deposits of the patient treated. In a preferred embodiment the botulinum neurotoxin is administered between 0.005 mm and 30 mm below the skin surface. In a more preferred embodiment the botulinum neurotoxin is injected between 5 mm and 20 mm below the skin surface. Generally, it is understood in the context of the present invention that the botulinum neurotoxin is not administered to the muscles in the treatment area of the patient.

In particular embodiments, the botulinum neurotoxin is administered to the lower limb (lower extremities) region and/or to the upper limb (upper extremities) region. The lower limbs comprise the following regions: buttock, thigh, knee, leg, ankle and foot. The upper limb is the region extending from the deltoid region up to and including the hand, including the arm, axilla and shoulder.

In particular embodiments, the botulinum neurotoxin is administered to a region selected from the group consisting of shin, calf, ankle, inner thighs and the region between hand and armpit. In a more specific embodiment the botulinum neurotoxin is administered to a region between wrist and elbow. In a further embodiment the botulinum neurotoxin for use is administered to the region between elbow and armpit. Particularly, the botulinum neurotoxin is administered in the context of the present invention to the lower legs and inner thighs.

In one preferred embodiment of the present invention, the maximum total dosage of the botulinum neurotoxin per treatment is about 400 U. In another embodiment of the present invention, the maximum total dosage of the botulinum neurotoxin is about 800 U. Particularly, the total dosage of the botulinum neurotoxin per treatment is between 2 and 800 U, preferably between 10 and 300 U, more particularly between 10 and 400 U, more particularly between 20 and 200 U.

Generally the botulinum neurotoxin is reconstituted prior use by dissolving the freeze dried botulinum neurotoxin preparation in an aqueous solution of physiological sterile saline. As used herein according to a preferred embodiment of the present invention, the botulinum neurotoxin is administered in a concentration of about 40 U/mL, i.e. a 100 U vial is reconstituted in about 2.5 mL sterile saline. In a further embodiment of the present invention, the botulinum neurotoxin is administered using a higher concentration than 40 U/mL. In particular embodiments of the present invention the botulinum neurotoxin is administered using a concentration between about 1000 U/mL and about 50 U/mL. In a preferred embodiment of the present invention the botulinum neurotoxin is administered using a concentration between about 200 U/mL and about 50 U/mL. In a preferred embodiment of the present invention the botulinum neurotoxin is administered using a concentration between 150 U/mL and about 50 U/mL. In a preferred embodiment of the present invention the botulinum neurotoxin is administered using a concentration between 100 U/mL and about 50 U/mL.

In a further embodiment of the present invention, the botulinum neurotoxin is administered using a lower concentration than 40 U/mL. In particular embodiments of the present invention the botulinum neurotoxin is administered using a concentration between about 35 U/mL and about 10 U/mL. In a preferred embodiment of the present invention the botulinum neurotoxin is administered using a concentration between about 25 U/mL and about 10 U/mL. After the administration, an additional massage of the tissue is recommended to distribute the fluid evenly within the tissue.

In vivo assays for assessing biological activity include the mouse LD50 assay and the ex vivo mouse hemidiaphragm assay as described by Pearce et al. (Pearce 1994, Toxicol. Appl. Pharmacol. 128: 69-77) and Dressler et al. (Dressler 2005, Mov. Disord. 20:1617-1619, Keller 2006, Neuroscience 139: 629-637) or a cell-based assay as described in WO2009/114748, WO2014/207109 or WO 2013/049508. The biological activity is commonly expressed in Mouse Units (U). As used herein, 1 U is the amount of neurotoxic component of the botulinum neurotoxin, which kills 50% of a specified mouse population after intraperitoneal injection, i.e. the mouse i.p. LD50. A particular useful method for determining the biological activity of a botulinum neurotoxin is a cell-based assay as it is disclosed for example in WO2009/114748, WO 2013/049508 or WO 2014/207109. The activity results obtained with such cell-based assays correspond to the activity values obtained in the mouse i.p. LD50 assay. Activity results obtained for Botulinum serotype A formulations like commercially available lncobotulinumtoxin A (Botulinumtoxin serotype A, without complexing proteins, Xeomin®, Merz Pharmaceuticals GmbH)) or Onabotulinumtoxin A (Botulinumtoxin serotype A, with complexing proteins, Botox®, Allergan Inc.) can be converted to values for other toxins using conversion rates known to the person skilled in the art. For example, the necessary dose of Abobotulinumtoxin A (Botulinumtoxin serotype A, with complexing proteins, Dysport®, Ipsen Biopharm Limited) can be determined by multiplication of the dose of lncobotulinumtoxin A or Onabotulinumtoxin A with a factor of 2.5 to 5. The dose for RimabotulinumtoxinB (Botulinumtoxin serotype B, Myobloc®, Solstice Neurosciences/US WorldMeds LLC) can be calculated by multiplication of the dose of lncobotulinumtoxin A or Onabotulinumtoxin A with a factor of 20 to 40.

Generally the botulinum toxin of the present invention is administered in a dosage of between 0.5 U and 50 U per injection site. In particular embodiments of the present invention, the botulinum neurotoxin is administered in a dosage of between 2 U and 5 U per injection site. In a preferred embodiment of the present the botulinum neurotoxin is administered in a dosage of between 0.5 U and 10 U per injection site. In case of fan-like injection technique the total dose for the area covered with the fan-like injection will be evenly distributed in the tissue.

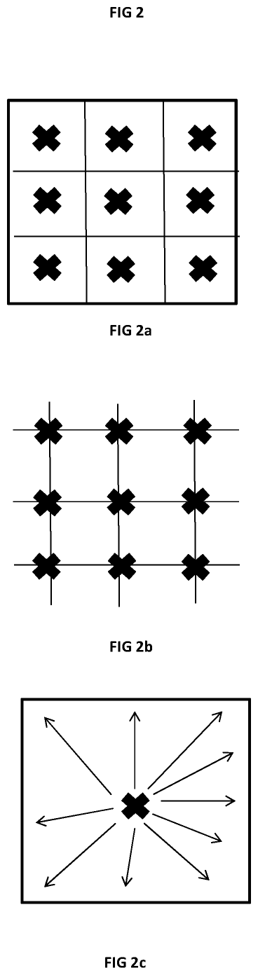

In particular embodiments of the present invention, the botulinum neurotoxin is administered to more than one injection site and the distance between two injections sites is about 1 to 2 cm. Generally, the botulinum neurotoxin is administered using between 2 to 20 injections per 10 cm². In a more preferred embodiment, the botulinum neurotoxin is administered using between 9-100 injections per 10 cm². Generally, the injections are evenly distributed in the target area using a regular chess-type pattern. In specific cases, the botulinum neurotoxin is administered using patterns which deviate from the regular chess-type pattern to address specific needs of the patients having the lipoedema. For example, when wishing to have the lowest possible number of injection points, a fan-like injection with blunt cannula (20-28 gauge) can be done. Three possible options for injection schemes are summarized in FIGS. 2a, 2b and 2c.

FIG. 2a: Injecting the botulinum neurotoxin into grid lumen.

FIG. 2b: Injecting the botulinum neurotoxin on grid crossings.

FIG. 2c: Fan-like application of the botulinum neurotoxin around central injection point.

In particular embodiments, the botulinum neurotoxin according to the invention is a botulinum neurotoxin complex.

In the context of the present invention, the terms “toxin complex” or “botulinum toxin complex” or “botulinum neurotoxin complex” are interchangeable and refer to a high molecular weight complex comprising the neurotoxic component of approximately 150 kDa and, in addition non-toxic proteins of Clostridium botulinum, including hemagglutinin and non-hemagglutinin proteins (Sakaguchi 1983; Sugiyama 1980). Botulinum toxins, when released from lysed Clostridium cultures are generally associated with other bacterial proteins, which together form of a toxin complex. This complex usually contains additional, so-called “non-toxic” proteins, which will be referred here to as “complexing proteins” or “bacterial proteins”. The complex of neurotoxic component and bacterial proteins is referred to as “Clostridium botulinum toxin complex” or “botulinum toxin complex”. The molecular weight of this complex may vary from about 300,000 to about 900,000 Da. It is commercially available as Botulinum toxin A protein complex, for example, under the tradename BOTOX/VISTABEL (Allergan Inc) or under the tradename DYSPORT/AZZALURE (Ipsen Ltd).

In the context of the present invention, the terms “neurotoxic component” or “neurotoxin component” as used throughout the specification, relates to the subunit of the botulinum toxin complex which has a neurotoxic activity and which has a molecular weight of approximately 150 kDa in serotype A. Unlike the toxin complex, the neurotoxic component in its isolated and pure form, i.e. devoid of any complexing Clostridium proteins, is acid labile and does not resist the aggressive environment in the gastrointestinal tract. The term “neurotoxic component” also includes the functional homologs found in the other serotypes of Clostridium botulinum.

In particular embodiments, the botulinum neurotoxin according to the invention is the neurotoxic component of a botulinum neurotoxin complex, wherein said neurotoxic component is devoid of any other protein component of the Clostridium botulinum neurotoxin complex.

In the context of the present invention, the term “devoid of any other protein component of the Clostridium botulinum neurotoxin complex” means without any non-toxic proteins of Clostridium botulinum, for example hemagglutinin proteins.

In the context of the present invention the term “opiate or opiate derivative” means an analgesic drug like, for example, morphine analogues or derivatives like fentanyl, alfentanil, codein, dihydrocodein, hydrocodone, oxycodone, hydromorphone, pethidine, remifentanil, sufentanil, dextropropoxyphene, tramadol, buprenorphine, nalbuphine and morphine.

In particular embodiments, the botulinum neurotoxin according to the invention is selected from the group of different serotypes including botulinum neurotoxin serotype A (BoNT/A), botulinum neurotoxin serotype B (BoNT/B), botulinum neurotoxin serotype C1 (BoNT/C1), botulinum neurotoxin serotype D (BoNT/D), botulinum neurotoxin serotype E (BoNT/E), botulinum neurotoxin serotype F (BoNT/F) or botulinum neurotoxin serotype G (BoNT/G). The botulinum neurotoxin and, in particular, its light chain and heavy chain are derivable from one of the antigenically different serotypes of botulinum neurotoxins indicated above. In an aspect, said light and heavy chain of the botulinum neurotoxin are the light and heavy chain of a botulinum neurotoxin selected from the group consisting of: BoNT/A, BoNT/B, BoNT/C1, BoNT/D, BoNT/E, BoNT/F, or BoNT/G. In another aspect, a polynucleotide encoding said botulinum neurotoxin of the present invention comprises a nucleic acid sequence as shown in SEQ ID NO: 1 (BoNT/A), SEQ ID NO: 3 (BoNT/B), SEQ ID NO: 5 (BoNT/C1), SEQ ID NO: 7 (BoNT/D), SEQ ID NO: 9 (BoNT/E), SEQ ID NO: 11 (BoNT/F), or SEQ ID NO: 13 (BoNT/G). Moreover, encompassed is, in an aspect, a polynucleotide comprising a nucleic acid sequence encoding an amino acid sequence as shown in any one of SEQ ID NO: 2 (BoNT/A), SEQ ID NO: 4 (BoNT/B), SEQ ID NO: 6 (BoNT/C1), SEQ ID NO: 8 (BoNT/D), SEQ ID NO: 10 (BoNT/E), SEQ ID NO: 12 (BoNT/F), or SEQ ID NO: 14 (BoNT/G). Further encompassed is in an aspect the means and methods of the present invention to produce a botulinum neurotoxin comprising or consisting of an amino acid sequence selected from the group consisting of: SEQ ID NO: 2 (BoNT/A), SEQ ID NO: 4 (BoNT/B), SEQ ID NO: 6 (BoNT/C1), SEQ ID NO: 8 (BoNT/D), SEQ ID NO: 10 (BoNT/E), SEQ ID NO: 12 (BoNT/F), and SEQ ID NO: 14 (BoNT/G).

In another aspect, the said polynucleotide encoding a botulinum neurotoxin of the present invention is a variant of the aforementioned polynucleotides comprising one or more nucleotide substitutions, deletions and/or additions which in still another aspect may result in a polypeptide having one or more amino acid substitutions, deletions and/or additions. Moreover, a variant polynucleotide of the invention shall in another aspect comprise a nucleic acid sequence variant being at least 40%, at least 50%, at least 60%, at least 70%, at least 75%, at least 80%, at least 85%, at least 90%, at least 95%, at least 96%, at least 97%, at least 98% or at least 99% identical to the nucleic acid sequence as shown in any one of SEQ ID NOs: 1, 3, 5, 7, 9, 11, 13 or 15 or a nucleic acid sequence variant which encodes an amino acid sequence being at least 40%, at least 50%, at least 60%, at least 70%, at least 75%, at least 80%, at least 85%, at least 90%, at least 95%, at least 96%, at least 97%, at least 98% or at least 99% identical to the amino acid sequence as shown in any one of SEQ ID NOs: 2, 4, 6, 8, 10, 12, 14, or 16. The term “identical” as used herein refers to sequence identity characterized by determining the number of identical amino acids between two nucleic acid sequences or two amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wis., USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. In an aspect, each of the aforementioned variant polynucleotides encodes a polypeptide retaining one or more and, in another aspect, all of the biological properties of the respective botulinum neurotoxin, i.e. the BoNT/A, BoNT/B, BoNT/C1, BoNT/D, BoNT/E, BoNT/F or BoNT/G. Those of skill in the art will appreciate that full biological activity is maintained only after proteolytic activation, even though it is conceivable that the unprocessed precursor can exert some biological functions or be partially active. “Biological properties” as used herein refers to (a) receptor binding, (b) internalization, (c) translocation across the endosomal membrane into the cytosol, and/or (d) endoproteolytic cleavage of proteins involved in synaptic vesicle membrane fusion. In a further aspect, the variant polynucleotides can encode botulinum neurotoxins having improved or altered biological properties, e.g., they may comprise cleavage sites which are improved for enzyme recognition or may be improved for receptor binding or any other property specified above.

In particular embodiments, the botulinum neurotoxin is administered together with at least one standard lipoedema therapy selected from compression therapy, movement therapy, weight reduction, breathing physiotherapy, functional lymphological rehabilitation and/or manual lymphatic drainage or combinations thereof. If a manual lymphatic drainage is used as combination treatment after the botulinum neurotoxin, it is recommended to have the manual lymphatic drainage only after at least three days after the botulinum neurotoxin administration.

In another aspect, the present invention relates to a pharmaceutical composition comprising a botulinum neurotoxin according to the invention for use in treating a disease or condition associated with lipoedema. For preparing a pharmaceutical preparation comprising a botulinum neurotoxin the neurotoxin can be formulated by various techniques dependent on the desired application purposes which are known in the art. For example, the (biologically active) botulinum neurotoxin can be used in combination with one or more pharmaceutically acceptable carriers as a pharmaceutical composition. The pharmaceutically acceptable carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatine, agar, pectin, acacia, magnesium stearate, stearic acid, hyaluronic acid (dry or diluted) and the like. Exemplary of liquid carriers are glycerol, phosphate buffered saline solution, water, emulsions, hyaluronic acid solution, various types of wetting agents, and the like. Suitable carriers comprise those mentioned above and others well known in the art, see, e.g., Remington's Pharmaceutical Sciences, Mack Publishing Company, Easton, Pa. In an aspect, the pharmaceutical composition can be dissolved in a diluent, prior to administration. The diluent is also selected so as not to affect the biological activity of the botulinum neurotoxin product. Examples of such diluents are distilled water, Ringer solution or physiological saline. In addition, the pharmaceutical composition or formulation may also include other carriers or non-toxic, non-therapeutic, non-immunogenic stabilizers and the like. Thus, the formulated botulinum neurotoxin product can be present, in an aspect, in liquid or lyophilized form. In an aspect, it can be present together with glycerol, protein stabilizers (HSA) or non-protein stabilizers such as polyvinyl pyrrolidone (PVP), hyaluronic acid or free amino acids. In an aspect, suitable non-proteinaceous stabilizers are disclosed in WO 2005/007185 or WO 2006/020208. A suitable formulation for HSA-stabilized formulation comprising a botulinum neurotoxin according to the present invention is for example disclosed in U.S. Pat. No. 8,398,998 B2. The formulated botulinum neurotoxin product may be used for human or animal therapy of various diseases or disorders in a therapeutically effective dose or for cosmetic purposes.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components, or groups thereof. The term “comprising” thus includes the more restrictive terms “consisting of” and “consisting essentially of”.

In particular embodiments, the pharmaceutical composition comprising a botulinum neurotoxin according to the invention is for use in treating pain associated with lipoedema.

In another aspect, the present invention relates to a method of treating a disease or condition associated with lipoedema, wherein the method comprises the administration of a therapeutically effective amount of a botulinum neurotoxin according to the invention.

In particular embodiments, the method according to the invention is treating pain associated with lipoedema.

### EXAMPLES

### Example 1: Treatment of Pain in Lipoedema

A 50 year old female patient suffering from lipoedema has pain at both distal lower limbs. The patient has been treated with compression therapy and movement therapy without success. The patient has stopped treatment with the oral opiate Tilidin due to side effects affecting her day to day life significantly without providing sufficient relief. The patient is evaluated for botulinum toxin therapy. After all appropriate examinations an injection scheme is developed and botulinum toxin serotype A free of complexing proteins (Xeomin®) is administered in a total dose of 400 U at both distal lower limbs using a chess-type injection scheme (i.e. an even distribution of the injection points) with 2 U botulinum neurotoxin per injection site. On re-evaluation after 2-4 weeks the symptomatology is improved, i.e the patient suffers significantly less pain. Adverse effects do not occur. The patient can start physiotherapy with further reduction of pain.

### Example 2: Treatment of Pain in Lipoedema

A 42 year old female suffering from lipoedema has pain in all four extremities. She declines opiate therapy due to drowsiness and does not profit from non-opiate pain remedies like Paracetamol or Ibuprofen. She receives botulinum toxin serotype A free of complexing proteins (Xeomin®) in a total dose of 800 U in both lower legs using a chess-type administration scheme using 4 U Xeomin® per injection site and her pain is dramatically reduced. After two cycles of Xeomin® therapy she has a liposuction on both legs and does not need any further post-surgical pain therapy.

1. A product comprising a botulinum neurotoxin for treating a disease or condition associated with lipoedema, wherein the botulinum neurotoxin is not administered in combination with an opiate or opiate derivative.

2. The product comprising a botulinum neurotoxin according to claim 1, wherein the disease or condition associated with lipoedema is pain.

3. The product comprising a botulinum neurotoxin according to claim 1 or 2, wherein the botulinum neurotoxin is administered by intradermal or subcutaneous injection.

4. The product comprising a botulinum toxin according to claim 1, wherein the toxin is administered to the lower limb and/or upper limb, optionally to a region selected from the group consisting of shin, calf, ankle, inner thighs and the region between wrist and armpit.

5. The product comprising a botulinum neurotoxin according to claim 1, wherein the botulinum neurotoxin is administered in a dosage of between 0.5 U and 50 U per injection site.

6. The product comprising a botulinum neurotoxin according to claim 1, wherein the maximum total dosage of the botulinum neurotoxin per treatment is about 400 U.

7. The product comprising a botulinum neurotoxin according to claim 1, wherein the botulinum neurotoxin is administered to more than one injection site and the distance between two injections sites is about 1 to 2 cm.

8. The product comprising a botulinum neurotoxin according to claim 1, wherein said botulinum neurotoxin is a botulinum neurotoxin complex.

9. The product comprising a botulinum neurotoxin according to claim 1, wherein said botulinum neurotoxin is the neurotoxic component of a botulinum neurotoxin complex, wherein said neurotoxic component is devoid of any other protein component of the Clostridium botulinum neurotoxin complex.

10. The product comprising a botulinum neurotoxin according to claim 1, wherein said botulinum neurotoxin is selected from the group of serotypes consisting of A, B, C1, D, E, F and G.

11. The product comprising a botulinum neurotoxin according to claim 1, wherein the botulinum neurotoxin is administered together with at least one standard lipoedema therapy selected from compression therapy, movement therapy, weight reduction, breathing physiotherapy, functional lymphological rehabilitation and/or manual lymphatic drainage or combinations thereof.

12. A pharmaceutical composition comprising a botulinum neurotoxin for treating a disease or condition associated with lipoedema, wherein the botulinum neurotoxin is not administered in combination with an opiate or opiate derivative.

13. The pharmaceutical composition according to claim 12, wherein the disease or condition associated with lipoedema is pain.

14. A method of treating a disease or condition associated with lipoedema, wherein the method comprises administering a therapeutically effective amount of a botulinum neurotoxin, wherein the botulinum neurotoxin is not administered in combination with an opiate or opiate derivative.

15. The method according to claim 14, wherein the disease or condition associated with lipoedema is pain.
